(19) United States
(12) Patent Application Publication
(10) Pub. No.: US 2020/0340998 A1
(43) Pub. Date: Oct. 29, 2020

(54) METHOD FOR DIAGNOSING CANCER, ASSESSING CANCER PROGNOSIS, MONITORING CANCER, OR ASSESSING EFFECTIVENESS OF CANCER TREATMENT

(71) Applicant: CHANG GUNG UNIVERSITY, Taoyuan City (TW)

(72) Inventors: Min-Hsien Wu, Taoyuan City (TW); Chia-Jung Liao, Taoyuan City (TW); Feng-Chung Hung, Taoyuan City (TW)

(21) Appl. No.: 16/680,552

(22) Filed: Nov. 12, 2019

(30) Foreign Application Priority Data
Apr. 23, 2019 (TW) 108114158

(51) Int. Cl. G01N 33/574; G01N 33/58; G01N 1/34; G01N 1/30

(52) U.S. Cl. CPC G01N 33/57492; G01N 1/30; G01N 1/34; G01N 33/582

(57) ABSTRACT

The present disclosure provides a method for diagnosing cancer, assessing cancer prognosis, monitoring cancer, or assessing effectiveness of cancer treatment. The present disclosure also provides a method for diagnosing cancer, assessing cancer prognosis, monitoring cancer, or assessing effectiveness of cancer treatment by using an atypical circulating tumor cell.

### CROSS-REFERENCE TO RELATED APPLICATION

This application claims priority of Taiwan patent application No. 108114158, filed on Apr. 23, 2019, the content of which is incorporated herein in its entirety by reference.

### BACKGROUND OF THE INVENTION

### 1. Field of the Invention

The present invention relates to a method for diagnosing cancer, assessing cancer prognosis, monitoring cancer, or assessing effectiveness of cancer treatment. The present invention also relates to a method for diagnosing cancer, assessing cancer prognosis, monitoring cancer, or assessing effectiveness of cancer treatment by using an atypical circulating tumor cell.

### 2. The Prior Art

Cancer metastasis is the leading cause of cancer-related death. Circulating tumor cells (CTCs), which have been confirmed since 1869, are cells that escape from the primary tumor site to the adjacent vasculature and subsequently present in the blood circulation. There is evidence that the presence of circulating tumor cells in the blood circulation is associated with cancer metastasis. Therefore, those skilled in the art have focused on studying circulating tumor cells to understand the mechanism of cancer metastasis. This research direction can stimulate the skilled artisan to develop new cancer treatment strategies.

In addition, in clinical applications, analysis of circulating tumor cells (considered as liquid tumor biopsy) can be used as a diagnostic or prognostic tool for monitoring cancer metastasis or therapeutic response, and guiding individualization treatment. In order to achieve these goals, it is necessary to isolate circulating tumor cells with high purity from blood samples to avoid as much as possible analysis interference caused by peripheral blood cells (mainly white blood cells).

However, circulating tumor cells are very rare in blood samples at a concentration of approximately one circulating tumor cell per 10⁵ to 10⁷ blood mononuclear cells. This phenomenon makes it difficult to isolate and purify circulating tumor cells, particularly difficult to isolate and purify circulating tumor cells with high purity. At present, there are various methods for isolating and purifying circulating tumor cells, which can be roughly classified into physical and biochemical methods. In general, the physical method for isolating circulating tumor cells (primarily filtration) is easy to perform and does not require labeling of harvested cells, but the purity of the cells is lower than that of the biochemical methods. In the biochemical methods, the immune cell isolation method (such as the method of immunomagnetic beads) is mainly used for the isolation and purification of circulating tumor cells. In this method, magnetic beads coupled to specific antibodies of surface biomarkers (mainly epithelial cell adhesion molecule (EpCAM) and cytokeratins (CKs)) of circulating tumor cells are commonly used for identifying and binding to circulating tumor cells. Magnetically labeled circulating tumor cells are isolated from peripheral cells by an applied magnetic field. Circulating tumor cell isolation according to this method is primarily used in current circulating tumor cell isolation or detection systems (e.g., CellSearch™ system, magnetically activated cell sorting system, or Dynabeads™)

Although the above-described methods for isolating circulating tumor cells have been present, there are still many problems to be overcome. One of the problems is that white blood cell contamination in purified and isolated circulating tumor cells is often unavoidable, which may affect the accuracy of subsequent circulating tumor cell analysis (especially gene expression analysis). This fact highlights the importance of isolating circulating tumor cells with high purity for subsequent high precision analysis. In addition to the purity of circulating tumor cells, there are some important biological issues that are needed for further consideration. As mentioned above, most of the methods for isolating or purifying circulating tumor cells rely primarily on the use of EpCAM or CKs to identify circulating tumor cells. However, circulating tumor cells (especially circulating tumor cells with high metastatic potential) may undergo epithelial-to-mesenchymal transition (EMT), which allows cells to acquire the characteristics necessary for metastasis, such as migration and invasion, anti-apoptosis, and cancer stem cell characteristics. These circulating tumor cells undergoing EMT may reduce the expression of genes encoding epithelial cell markers such as EpCAM and CKs. In this regard, if a conventional method for isolating circulating tumor cells (relied on EpCAM and CKs-based positive selection) is used, these circulating tumor cells that undergo EMT and are clinically highly associated with cancer metastasis may be missed.

Therefore, those skilled in the art are in urgent need of developing novel methods for purifying, isolating and analyzing atypical circulating tumor cells (i.e., circulating tumor cells that do not express typical circulating tumor cell markers such as EpCAM and CKs) and uses of the atypical circulating tumor cells to overcome the disadvantages of the prior art and to benefit a large group of people in need thereof.

### SUMMARY OF THE INVENTION

A primary objective of the present invention is to provide a method for diagnosing cancer, assessing cancer prognosis, monitoring cancer, or assessing effectiveness of cancer treatment, comprising the following steps: (a) providing a whole blood from a subject; (b) performing a treatment on the whole blood to remove a plurality of red blood cells and a plurality of platelets to obtain a treated sample; (c) negatively selecting the treated sample using a blood cell depletion method to remove at least one blood cell positive for a blood cell surface protein to obtain a negatively selected cell population; (d) performing an immunofluorescence staining on the negatively selected cell population to identify a plurality of subpopulations of cells in the negatively selected cell population, wherein each of the plurality of subpopulations of cells comprises the at least one white blood cell and at least one non-leukocyte nucleated cell, and the at least one non-leukocyte nucleated cell comprises a typical circulating tumor cell negative for the blood cell surface protein and positive for a circulating tumor cell biomarker and an atypical circulating tumor cell; and (e) analyzing, identifying, measuring, and purifying the plurality of subpopulations of cells using a single cell analysis technique, and excluding blood cells and the typical circulating tumor cell by the blood cell surface protein and the circulating tumor cell biomarker to obtain the atypical circulating tumor cell and its quantitative information; wherein when an amount or genetic information of the atypical circulating tumor cell of the subject is greater than a cut-off value, a high-risk group suffering from cancer, cancer recurrence, poor effectiveness of cancer treatment, or poor prognosis of cancer is determined, and the cut-off value is a value obtained by a statistical analysis after a clinical trial.

According to an embodiment of the present invention, the blood cell surface protein is selected from the group consisting of CD3, CD4, CD8, CD11b, CD11c, CD14, CD19, CD20, CD33, CD34, CD41, CD45, CD56, CD61, CD62, CD66b, CD68, CD123 , CD146, Gly A, and any combination thereof.

According to an embodiment of the present invention, the circulating tumor cell biomarker is selected from the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), MET, and any combination thereof.

According to an embodiment of the present invention, the cancer is a liver cancer, a lung cancer, a colorectal cancer, a breast cancer, a nasopharyngeal cancer, a prostate cancer, an esophageal cancer, a pancreatic cancer, a skin cancer, a thyroid cancer, a stomach cancer, a kidney cancer, a gallbladder cancer, an ovarian cancer, a cervical cancer, a bone cancer, a brain cancer, or a head and neck cancer.

According to an embodiment of the present invention, the single cell analysis technique is selected from the group consisting of an immunofluorescence staining, a flow cytometry, a fluorescence microscopy, a microfluidic biochip system of optically-induced dielectrophoresis force, and any combination thereof.

According to an embodiment of the present invention, the atypical circulating tumor cell is in a free form.

Another objective of the present invention is to provide a method for diagnosing cancer, assessing cancer prognosis, monitoring cancer, or assessing effectiveness of cancer treatment by using at least one atypical circulating tumor cell negative for a blood cell surface protein and negative for a circulating tumor cell biomarker, wherein the at least one atypical circulating tumor cell is purified and isolated from a whole blood of a subject, and when an amount or genetic information of the at least one atypical circulating tumor cell of the subject is greater than a cut-off value, a high-risk group suffering from cancer, cancer recurrence, poor effectiveness of cancer treatment, or poor prognosis of cancer is determined, wherein the cut-off value is a value obtained by a statistical analysis after a clinical trial.

According to an embodiment of the present invention, the blood cell surface protein is selected from the group consisting of CD3, CD4, CD8, CD11b, CD11c, CD14, CD19, CD20, CD33, CD34, CD41, CD45, CD56, CD61, CD62, CD66b, CD68, CD123 , CD146, Gly A, and any combination thereof.

According to an embodiment of the present invention, the circulating tumor cell biomarker is selected from the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), MET, and any combination thereof.

According to an embodiment of the present invention, the cancer is a liver cancer, a lung cancer, a colorectal cancer, a breast cancer, a nasopharyngeal cancer, a prostate cancer, an esophageal cancer, a pancreatic cancer, a skin cancer, a thyroid cancer, a stomach cancer, a kidney cancer, a gallbladder cancer, an ovarian cancer, a cervical cancer, a bone cancer, a brain cancer, or a head and neck cancer.

According to an embodiment of the present invention, the at least one atypical circulating tumor cell is in a free form.

In summary, the effects of the present invention are as follows: high throughput, high purity and high recovery rate, no selection bias, successful purification, isolation and analysis of atypical circulating tumor cells. Through the present invention, multiple parameters can be recovered from a single sample for clinical diagnosis of cancer, assessing cancer prognosis, monitoring cancer, and assessing effectiveness of cancer treatment. Multi-parameter simultaneous analysis can improve the sensitivity and accuracy of clinical applications and become an important basis for the implementation of precision medicine. Therefore, the present invention has important application value in both clinical and basic research.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a schematic diagram of identification and analysis of CD45-negative and EpCAM-negative cells.

FIG. 2 is a schematic diagram showing the quantitative analysis of (A) typical circulating tumor cells; and (B) CD45-negative and EpCAM-negative cells in 22 healthy subjects and 39 cancer patients (including liver cancer, lung cancer, nasopharyngeal cancer, prostate cancer, esophageal cancer, pancreatic cancer, and head and neck cancer).

FIG. 3 is a schematic diagram showing the quantitative analysis of (A) typical circulating tumor cells; and (B) CD45-negative and EpCAM-negative cells in 22 healthy subjects and 27 head and neck cancer patients.

FIG. 4 is a data diagram showing the survival analysis of 22 healthy subjects and 27 head and neck cancer patients.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

As used herein, the term “circulating tumor cell (CTC)” is intended to encompass any rare tumor cell present in a biological sample associated with cancer.

As used herein, the term “magnetic activated cell-sorting” refers to a method of cell sorting using immunomagnetic beads. The surface of the magnetic beads is coated with an immunoreactive antibody, which can react with an antigen on a target cell for antigen-antibody reaction. When these cells combined with magnetic beads are placed under a magnetic field, they are separated from other unbound cells. The magnetic beads with magnetic fields lose their magnetic properties immediately after they are detached from the magnetic field, thereby selecting or removing the labeled cells to achieve the purpose of obtaining positive or negative cells.

### EXAMPLE 1

### Whole Blood Sample Treatment and CD45-Negative and EpCAM-Negative Cell Quantitative Methods

In this example, the experiment was approved by the Institutional Review Board of the Chang Gung Memorial Hospital. All blood sample donors received informed consent (approval number: 201601081B0) and all methods were performed in accordance with the guidelines for clinical trials.

First, 4 mL of a whole blood sample from a subject was provided, and then the blood cells in the 4 mL of the whole blood sample were removed. Red blood cell lysis buffer was used, in which 1 L of red blood cell lysis buffer contains 8.26 g of NH₄Cl, 1.19 g of NaHCO₃, 200 μL of 0.5 M, pH 8 of EDTA (ethylenediaminetetraacetic acid), and the final pH is 7.3. The volume ratio of the whole blood sample to the red blood cell lysis buffer is 1:10, the reaction is not more than 10 minutes, and the supernatant is removed by centrifugation. The platelets were removed by centrifugation at 100 to 200×g to obtain a treated sample. The treated sample was negatively selected using a blood cell depletion method to remove at least one blood cell that is positive for at last one blood cell surface protein (e.g., CD3, CD4, CD8, CD11b, CD11c, CD14, CD19, CD20, CD33, CD34, CD41, CD45, CD56, CD61, CD62, CD66b, CD68, CD123, CD146, Gly A, and any combination thereof). In this example, CD45-positive white blood cells were removed according to the procedure of the EasySep™ CD45 depletion kit (StemCell Technologies, Vancouver, BC, Canada) to obtain a negatively selected cell population.

Subsequently, immunofluorescence staining was performed on the negatively selected cell population to identify a plurality of subpopulations of cells in the negatively selected cell population, wherein each of the plurality of subpopulations of cells comprises the at least one white blood cell and at least one non-leukocyte nucleated cell, and the at least one non-leukocyte nucleated cell comprises a typical circulating tumor cell which is negative for the blood cell surface protein and positive for a circulating tumor cell biomarker (e.g., epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), MET, and any combination thereof), and an atypical circulating tumor cell which is negative for the blood cell surface protein and negative for the circulating tumor cell biomarker.

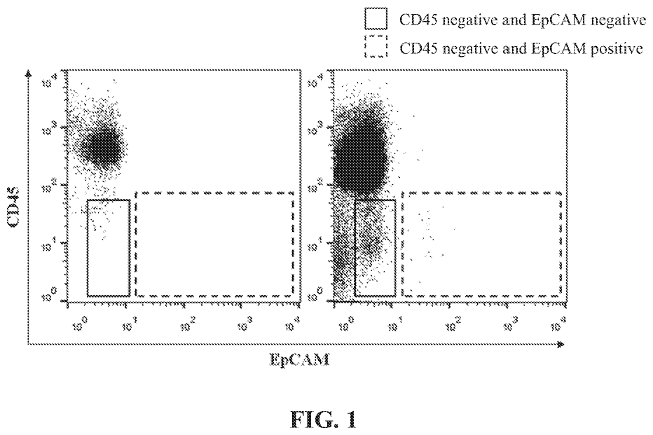

In this example, the immunofluorescence staining process is as follows: the nuclei were stained with a nuclear dye. White blood cells and typical circulating tumor cells were labeled with fluorescent material-conjugated antibodies, and the labeled target proteins are CD45 and EpCAM, respectively, or other cell marker-specific antibodies that can recognize these two types of cell. After the staining was completed, the excess antibody was washed away with PBS to complete the staining step. The single cell analysis technique, such as flow cytometry and the optically-induced dielectrophoresis (ODEP)-based microfluidic chip system, was used to identify, analyse, quantify, purify, and isolate the subpopulations of cells after immunofluoresent staining Blood cells and the typical circulating tumor cell were excluded by the blood cell protein markers and the circulating tumor cell biomarkers to obtain the CD45-negative and EpCAM-negative atypical circulating tumor cells and their quantitative information, as shown in FIG. 1.

### EXAMPLE 2

### The Specificity and Sensitivity of Using the CD45-Negative and EpCAM-Negative Cells to Identify Cancer Patients

In this example, the experiment was approved by the Institutional Review Board of the Chang Gung Memorial Hospital. All blood sample donors received informed consent (approval number: 201601081B0) and all methods were performed in accordance with the guidelines for clinical trials.

First, CD45-negative and EpCAM-negative atypical circulating tumor cells and their quantitative information were obtained according to the procedure described in Example 1. A cut-off value is set according to the mean, median, or the receiver operating characteristic curve (ROC curve) of analytical populations. In this example, the cut-off value is set according to the ROC curve of analytical populations. Thereafter, statistical analysis is used to calculate the specificity and sensitivity. Specificity is the proportion of healthy subjects diagnosed as negative (true negative÷total number of healthy subjects (true negative+false positive)). Sensitivity is the proportion of cancer patients diagnosed as positive (true positive÷total number of cancer patients (true positive+false negative)). In addition, when the amount of the atypical circulating tumor cell of the subject is greater than the cut-off value, a high-risk group suffering from cancer, cancer recurrence, poor effectiveness of cancer treatment, or poor prognosis of cancer is determined. In this example, the cancer was exemplified by head and neck cancer, and the results are shown in Table 1.

TABLE 1

| | Present invention¹ | | CellSearch ®² |
|---|---|---|---|
| | Circulating tumor | CD45 negative and | Circulating tumor |
| | cell | EpCAM negative cell | cell |
| | (Cut-off | (Cut-off | (Cut-off |
| | value = 2) | value = 400) | value = 1) |
| Sensitivity | 29.6 | 33.3 | 24.6 |
| (%) | | | |
| Specificity | 100 | 100 | 100 |
| (%) | | | |

Note
¹22 healthy subjects, and 27 patients with advanced (third to fourth stage) head and neck cancer.
Note
²209 healthy subjects, and 852 patients with advanced (third to fourth stage) head and neck cancer.

The result of this example shows that the method of the present invention has higher sensitivity (specificity=100%) for identifying cancer patients by comparing to the conventional CellSearch® system.

### EXAMPLE 3

### Clinical Significance of the Number of Typical Circulating Tumor Cells and Number of CD45-Negative and EpCAM-Negative Cells

In this example, the experiment was approved by the Institutional Review Board of the Chang Gung Memorial Hospital. All blood sample donors received informed consent (approval number: 201601081B0) and all methods were performed in accordance with the guidelines for clinical trials.

First, CD45-negative and EpCAM-negative atypical circulating tumor cells and their quantitative information were obtained according to the procedure described in Example 1. The average numbers of cell populations in healthy subjects and cancer patients were compared by the statistical method. The statistical method is Mann-Whitney U test. The P value less than 0.05 is considered as statistically significant.

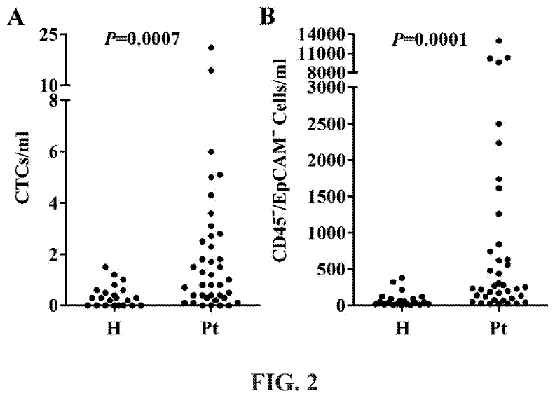

FIG. 2 is a schematic diagram showing the quantitative analysis of (A) typical circulating tumor cells; and (B) CD45-negative and EpCAM-negative cells in 22 healthy subjects and 39 cancer patients (including liver cancer, lung cancer, nasopharyngeal cancer, prostate cancer, esophageal cancer, pancreatic cancer, and head and neck cancer), wherein H indicates a healthy subject, and Pt indicates a cancer patient. As shown in FIG. 2, the numbers of both cell populations in cancer patients are significantly higher than those in healthy subjects.

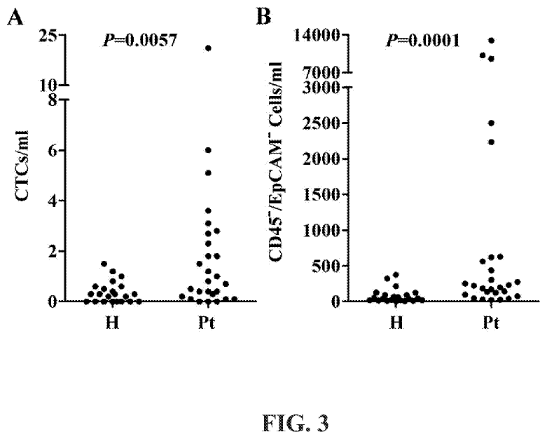

FIG. 3 is a schematic diagram showing the quantitative analysis of (A) typical circulating tumor cells; and (B) CD45-negative and EpCAM-negative cells in 22 healthy subjects and 27 head and neck cancer patients, wherein H indicates a healthy subject, and Pt indicates a head and neck cancer patient. As shown in FIG. 3, the numbers of both cell populations in head and neck cancer patients are significantly higher than those in healthy subjects. Therefore, the method of the present invention can be applied to identify the individuals who were at high risk for suffering from cancers.

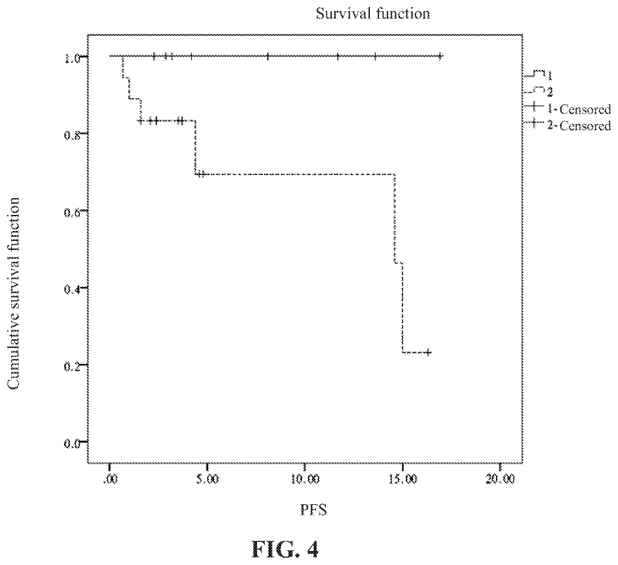

The head and neck cancer patients are divided into high and low cell number groups according to the two cell populations. Survival analysis (i.e., Kaplan-Meier analysis) was performed on the high and low cell number groups. The blood samples of subjects were collected before treatment; and the first instance of cancer-specific disease progression or death after treatment was defined as event of survival analysis. The result is shown in FIG. 4. FIG. 4 is a data diagram showing the survival analysis of 22 healthy subjects and 27 head and neck cancer patients, wherein PFS indicates progression-free survival in months. As shown in FIG. 4, progression-free survival time of the group of high CD45-negative and EpCAM-negative cell numbers (dashed line) is shorter than that in the group of low CD45-negative and EpCAM-negative cell numbers (solid line). The result of this example demonstrates that the method of the present invention is indeed applicable for assessing cancer prognosis.

### EXAMPLE 4

### CD45-Negative and EpCAM-Negative Cell Populations Isolated From Blood Samples of Head and Neck Cancer Patients and Analysis of Clinical Significance Regarding Gene Expression of CD45-Negative and EpCAM-Negative Cell Populations

In this example, the experiment was approved by the Institutional Review Board of the Chang Gung Memorial Hospital. All blood sample donors received informed consent (approval number: 201601081B0) and all methods were performed in accordance with the guidelines for clinical trials.

In conventional CTC-related studies, the cellular proteins EpCAM and CKs are predominately used as biomarkers to identify CTCs. However, growing evidence has suggested that the use of these biomarkers to identify CTCs is not sufficient due to the heterogeneous characteristics of CTCs. It is well recognized that the CTCs with a highly metastatic nature might undergo EMT, after which their expression of EpCAM and CKs is downregulated. Therefore, these cells are generally ignored in the conventional positive selection-based CTC (expressing EpCAM and CKs) isolation schemes. As a result, to development a strategy to comprehensively isolate those more clinically meaningful cells, e.g. circulating tumor cells which underwent EMT process, is important. Negative selection can avoid the problem of selection bias in positive selection. Moreover, it was discovered that after negative selection of blood samples from cancer patients, the number of CD45neg/EpCAMneg nucleated cells in the blood samples of cancer patients is significantly higher than that of healthy donors, and the number is related to the prognosis of patients (as shown in FIGS. 2-4).

To explore the clinical significance of gene expression of the CD45neg/EpCAMneg cell populations in blood samples of cancer patients, the blood samples (8 mL) were obtained from head and neck cancer patients (n=7), and treated according to the method in Example 1 to isolate CD45-positive white blood cells and CD45-negative and EpCAM-negative cell populations. The transcript differences between the two cell populations were analyzed by the next generation sequencing technique. After analysis, the genes unique to CD45-negative and EpCAM-negative cells were selected, and the result of the next generation sequencing was verified by real-time polymerase chain reaction.

Briefly, after purification and isolation of CD45-negative and EpCAM-negative cells, the cells were treated with the Ovation Solo RNA-Seq system (NuGEN Technologies, Inc.) to prepare a desired gene pool for sequencing, followed by performing sequencing by the Illumina HiSeq 4000 sequencing system. The sequencing result was subjected to quality analysis (Quality Value≥20), trimming, sequence mapping with the reference sequence (Hg19), calculation of transcript per million (TPM), comparison, and analysis to select genes unique to CD45-negative and EpCAM-negative cells. Afterward, TaqMan-based detection was carried to confirm the target gene expression levels of the isolated cells. In this example, the TaqMan kit (Hs01128573_g1) for the gene encoding thioredoxin related transmembrane protein 2 (TMX2) was purchased from Thermo Fisher Scientific, and the TaqMan assays (kit serial number: Hs01128573_g1) were performed according to the manufacturer's instructions. The β-2-microglobulin serves as the internal control gene in this example.

The next generation sequencing result of this example shows that TMX2 is one of the genes uniquely expressed in CD45-negative and EpCAM-negative atypical circulating tumor cells (TPM=7.78; and TPM=0 in white blood cells). After real-time PCR verification, the expression level of TMX2 gene in CD45-negative and EpCAM-negative atypical circulating tumor cells (17.25±30.79) is indeed higher than that in white blood cells (2.86±2.52). The result is shown in Table 2.

TABLE 2

| | | Transcript Per | |
|---|---|---|---|
| Gene | Cell type | Million (TPM) | real-time PCR² |
| TMX2 | White blood cell | 0 | 2.86 ± 2.52 |
| | Atypical circulating tumor | 7.48 | 17.25 ± 30.79 |
| | cell¹ | | |

Note
¹CD45-negative and EpCAM-negative nucleated cells in this example
Note
²relative expression level (ΔΔCq)

According to the expression level of TMX2 gene in CD45-negative and EpCAM-negative atypical circulating tumor cells, patients were divided into TMX2 low-expression and high-expression groups (cut-off value is the median), and the differences of the progression-free survival of the disease in the two groups were compared. The result is shown in Table 3.

TABLE 3

| | | Median | Half-year |
|---|---|---|---|
| Gene | Patient group | survival¹ | survival rate¹ |
| TMX2 | Cancer patient with | 11.4 | 80% |
| | low expression level | | |
| | Cancer patient with | 3.5 | 33% |
| | high expression level | | |

Note
¹progression-free survival of disease in months

As shown in Table 3, the median survival (3.5 months) and the half-year survival rate (33%) of patients with high TMX2 gene expression level are lower than those of patients with low TMX2 gene expression level (the median survival is 11.4 months, and the half-year survival rate is 80%). This result is in correspondence with previous studies that overexpression of the TMX2 gene is associated with the prognosis of liver cancer and head and neck cancer. This result confirms that the genetic information from CD45-negative and EpCAM-negative atypical circulating tumor cells does have potential for use in diagnosing cancer, assessing cancer prognosis, monitoring cancer, or assessing effectiveness of cancer treatment.

In summary, the effects of the present invention are as follows: high throughput, high purity and high recovery rate, no selection bias, successful purification, isolation and analysis of atypical circulating tumor cells. Through the present invention, multiple parameters can be recovered from a single sample for clinical diagnosis of cancer, assessing cancer prognosis, monitoring cancer, and assessing effectiveness of cancer treatment. Multi-parameter simultaneous analysis can improve the sensitivity and accuracy of clinical applications and become an important basis for the implementation of precision medicine. Therefore, the present invention has important application value in both clinical and basic research.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for diagnosing cancer, assessing cancer prognosis, monitoring cancer, or assessing effectiveness of cancer treatment, comprising the following steps:
(a) providing a whole blood from a subject;
(b) performing a treatment on the whole blood to remove a plurality of red blood cells and a plurality of platelets to obtain a treated sample;
(c) negatively selecting the treated sample using a blood cell depletion method to remove at least one blood cell positive for a blood cell surface protein to obtain a negatively selected cell population;
(d) performing an immunofluorescence staining on the negatively selected cell population to identify a plurality of subpopulations of cells in the negatively selected cell population, wherein each of the plurality of subpopulations of cells comprises the at least one white blood cell and at least one non-leukocyte nucleated cell, and the at least one non-leukocyte nucleated cell comprises a typical circulating tumor cell negative for the blood cell surface protein and positive for a circulating tumor cell biomarker and an atypical circulating tumor cell; and
(e) analyzing, identifying, measuring, and purifying the plurality of subpopulations of cells using a single cell analysis technique, and excluding blood cells and the typical circulating tumor cell by the blood cell surface protein and the circulating tumor cell biomarker to obtain the atypical circulating tumor cell and its quantitative information;
wherein when an amount or genetic information of the atypical circulating tumor cell of the subject is greater than a cut-off value, a high-risk group suffering from cancer, cancer recurrence, poor effectiveness of cancer treatment, or poor prognosis of cancer is determined, and the cut-off value is a value obtained by a statistical analysis after a clinical trial.

2. The method according to claim 1, wherein the blood cell surface protein is selected from the group consisting of CD3, CD4, CD8, CD11b, CD11c, CD14, CD19, CD20, CD33, CD34, CD41, CD45, CD56, CD61, CD62, CD66b, CD68, CD123 , CD146, Gly A, and any combination thereof.

3. The method according to claim 1, wherein the circulating tumor cell biomarker is selected from the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), MET, and any combination thereof.

4. The method according to claim 1, wherein the cancer is a liver cancer, a lung cancer, a colorectal cancer, a breast cancer, a nasopharyngeal cancer, a prostate cancer, an esophageal cancer, a pancreatic cancer, a skin cancer, a thyroid cancer, a stomach cancer, a kidney cancer, a gallbladder cancer, an ovarian cancer, a cervical cancer, a bone cancer, a brain cancer, or a head and neck cancer.

5. The method according to claim 1, wherein the single cell analysis technique is selected from the group consisting of an immunofluorescence staining, a flow cytometry, a fluorescence microscopy, a microfluidic biochip system of optically-induced dielectrophoresis force, and any combination thereof.

6. The method according to claim 1, wherein the atypical circulating tumor cell is in a free form.

7. A method for diagnosing cancer, assessing cancer prognosis, monitoring cancer, or assessing effectiveness of cancer treatment by using at least one atypical circulating tumor cell negative for a blood cell surface protein and negative for a circulating tumor cell biomarker, wherein the at least one atypical circulating tumor cell is purified and isolated from a whole blood of a subject, and when an amount or genetic information of the at least one atypical circulating tumor cell of the subject is greater than a cut-off value, a high-risk group suffering from cancer, cancer recurrence, poor effectiveness of cancer treatment, or poor prognosis of cancer is determined, wherein the cut-off value is a value obtained by a statistical analysis after a clinical trial.

8. The method according to claim 7, wherein the blood cell surface protein is selected from the group consisting of CD3, CD4, CD8, CD11b, CD11c, CD14, CD19, CD20, CD33, CD34, CD41, CD45, CD56, CD61, CD62, CD66b, CD68, CD123 , CD146, Gly A, and any combination thereof.

9. The method according to claim 7, wherein the circulating tumor cell biomarker is selected from the group consisting of epithelial cell adhesion molecule (EpCAM), cytokeratins (CKs), epidermal growth factor receptor (EGFR), CD44, CD24, vimentin, mucin 1 (Muc-1), E-cadherin, N-cadherin, Ras, human epidermal growth factor receptor 2 (Her2), MET, and any combination thereof.

10. The method according to claim 7, wherein the cancer is a liver cancer, a lung cancer, a colorectal cancer, a breast cancer, a nasopharyngeal cancer, a prostate cancer, an esophageal cancer, a pancreatic cancer, a skin cancer, a thyroid cancer, a stomach cancer, a kidney cancer, a gallbladder cancer, an ovarian cancer, a cervical cancer, a bone cancer, a brain cancer, or a head and neck cancer.

11. The method according to claim 7, wherein the at least one atypical circulating tumor cell is in a free form.
